(19) United States
(12) Patent Application Publication
(10) Pub. No.: US 2010/0087640 A1
(43) Pub. Date: Apr. 8, 2010

(54) Method for production of aryl-substituted annelated pyrimidines

(75) Inventors: Bernd Wolf, Fussgonheim (DE); Volker Maywald, Ludwigshafen (DE); Michael Keil, Freinsheim (DE); Manuel Budich, Bohl-lggelheim (DE); Michael Rack, Eppelheim (DE); Manfred Ehresmann, Ludwigshafen (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/521,877

(22) PCT Filed: Jan. 10, 2008
(86) PCT No.: PCT/EP2008/050254

§ 371 Date: Jul. 1, 2009

(30) Foreign Application Priority Data
Jan. 11, 2007 (EP) 07100430.3

(51) Int. Cl. C07D 487/04

(52) U.S. Cl. 544/263; 544/282

(57) ABSTRACT

The present invention relates to a process for preparing aryl-substituted fused pyrimidines of the general formula (I)

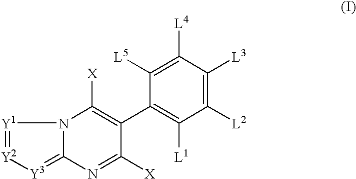

in which L¹ to L⁵ are H, halogen, CN, NO₂, C₁-C₄-alkyl, C₁-C₄-haloalkyl, C₁-C₄-alkoxy, C₁-C₄-haloalkoxy etc.; Y¹ to Y³ are C—R^{Y} or N; R^{Y} is H or optionally substituted C₁-C₄-alkyl or two adjacent R^{Y} together form a ring; X is OH, Cl or Br; which comprises
(i) the reaction of a 2-phenylmalonate with a compound (III) or a tautomer thereof,

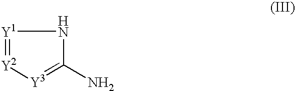

in the presence of a suitable base, where the alcohol, released during the reaction, of the formula R—OH is continuously removed from the reaction mixture under reduced pressure; giving a compound of the formula (I) or a salt thereof in which X is OH, and, if X in the compounds of the general formula (I) is chlorine or bromine,
(ii) the reaction of the compounds of the formula (I) obtained in step (i) or the salts with a halogenating agent.

The present invention relates to a process for preparing aryl-substituted fused pyrimidines which comprises reacting a 2-phenylmalonate in the presence of a suitable base and a heterocyclic amine, and reacting the dihydroxy-substituted compounds obtained in this manner with a halogenating agent.

Aryl-substituted fused pyrimidines, especially 5,7-dihalo- and 5,7-dihydroxy-6-aryl-1,2,4-triazolo[1,5-a]pyrimidines, are useful building blocks for the preparation of a large number of agrochemical and pharmaceutical compounds. They are, for example, key building blocks in the synthesis of fungicidal triazolopyrimidine derivatives as described, for example, in EP 0 550 113, EP 0 782 997, EP 0 770 615 or WO 98/46607.

EP 0 550 113 and EP 0 782 997 describe the preparation of 6-aryl-5,7-dihalo-1,2,4-triazolo[1,5-a]pyrimidines of the formula below

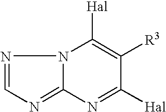

by reacting the corresponding 5,7-dihydroxy-substituted compounds with a halogenating agent. The 5,7-dihalotriazolopyrimidines obtained in this manner are reacted with ammonia or amines to give 7-aminotriazolopyrimidines. The 5,7-dihydroxy-substituted 1,2,4-triazolo[1,5-a]pyrimidines are provided from malonic esters and 3-amino-1,2,4-triazole.

EP 0 770 615 describes a process for preparing 5,7-dihalo-1,2,4-triazolo[1,5-a]pyrimidines and 5,7-dihaloimidazopyrimidines wherein in a first step, a malonic ester is reacted with a heterocyclic amine at a temperature of at least 100° C. The 5,7-dihydroxy-substituted triazolo- or imidazopyrimidines obtained in this manner or the salts thereof, formed as intermediates, are reacted at a temperature of at least 100° C. with at least two equivalents of a halogenating agent to give the 5,7-dihalo-1,2,4-triazolo[1,5-a]pyrimidines or 5,7-dihaloimidazopyrimidines.

The processes, known from the prior art, for preparing aryl-substituted fused pyrimidines are not entirely satisfactory with respect to the yields and product purities that can be obtained.

Accordingly, it is an object of the present invention to provide a process which affords aryl-substituted fused pyrimidines in high yield and purity.

Surprisingly, it has been found that this object is achieved by a process where a 2-phenylmalonate is reacted in the presence of a suitable base with a heteroaromatic 2-aminoazole, where the alcohol released during the reaction from the malonate is removed under reduced pressure from the reaction mixture. Surprisingly, it has also been found that fused 6-aryl-5,7-dihydroxypyrimidine compounds can be converted by reaction with a halogenating agent, in particular in the presence of an excess of the halogenating agent, into the corresponding fused 6-aryl-5,7-dihalopyrimidine compounds with particularly good yields and in high purity.

Accordingly, the present invention provides a process for preparing aryl-substituted fused pyrimidines of the general formula (I)

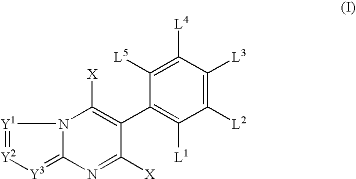

in which
L¹, L², L³, L⁴ and L⁵ independently of one another are hydrogen, halogen, cyano, nitro, C₁-C₄-alkyl, C₁-C₄-haloalkyl, C₁-C₄-alkoxy, C₁-C₂-haloalkoxy, C₁-C₄-alkylcarbonyl, C₁-C₄-haloalkylcarbonyl, C₁-C₄-alkoxycarbonyl, C₁-C₄-alkylaminocarbonyl or di-(C₁-C₄-alkyl)aminocarbonyl and
Y¹, Y², Y³ independently of one another are C—R^{Y} or N,
   where the substituents R^{Y} independently of one another are selected from the group consisting of hydrogen and C₁-C₄-alkyl which is optionally mono- or polysubstituted by halogen, cyano, nitro, C₁-C₄-alkoxy, C₁-C₂-haloalkoxy, C₁-C₄-alkylaminocarbonyl or di-(C₁-C₄-alkyl)aminocarbonyl; or
   where two adjacent substituents R^{Y} together with the atoms to which they are attached form an aromatic or partially saturated, optionally substituted 5- to 7-membered ring; and
X is hydroxyl, chlorine or bromine;
   which comprises
(i) the reaction of a 2-phenylmalonate of the general formula (II),

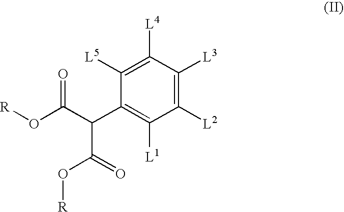

in which R is C₁-C₈-alkyl and the substituents L¹, L², L³, L⁴ and L⁵ have one of the meanings given above,
with a heterocyclic compound of the general formula (III) or a tautomer thereof,

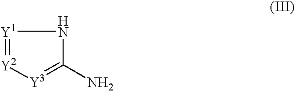

in the presence of a suitable base,
where the alcohol, released during the reaction, of the formula R—OH in which R is as defined above is continuously removed from the reaction mixture under reduced pressure;
giving a compound of the formula (I) or a salt thereof in which X is OH,
and, if X in the compounds of the general formula (I) is chlorine or bromine,
(ii) the reaction of the compounds of the formula (I) obtained in step (i) in which X is OH or the salts with a halogenating agent.

The terms used in the definition of the substituents for organic groups are, like, for example, the term “halogen”, collective terms which represent the individual members of these groups of organic moieties. In the particular case, the prefix Cₓ—C_{y} denotes the number of possible carbon atoms.

The term “halogen” denotes in each case fluorine, chlorine, bromine or iodine, especially fluorine, chlorine or bromine, in particular fluorine.

The term “C₁-C₄-alkyl”, as used herein and in the terms C₁-C₄-alkylcarbonyl, C₁-C₄-alkylaminocarbonyl and di(C₁-C₄-alkyl)aminocarbonyl, denotes a saturated straight-chain or branched hydrocarbon group comprising 1 to 4 carbon atoms, for example ethyl, propyl, 1-methylethyl, butyl, 1-methylpropyl, 2-methylpropyl or 1,1-dimethylethyl.

The term “C₁-C₄-haloalkyl”, as used herein and in the haloalkyl moieties of C₁-C₄-haloalkoxy and C₁-C₄-haloalkylcarbonyl, describes straight-chain or branched alkyl groups having 1 to 4 carbon atoms, where some or all of the hydrogen atoms of these groups are replaced by halogen atoms, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, etc.

The term “C₁-C₄-alkoxy”, as used herein, describes straight-chain or branched saturated alkyl groups comprising 1 to 4 carbon atoms, which groups are attached via an oxygen atom. Examples for C₁-C₄-alkoxy comprise, for example, methoxy, ethoxy, OCH₂—C₂H₅, OCH(CH₃)₂, n-butoxy, OCH(CH₃)—C₂H₅, OCH₂—CH(CH₃)₂, OC(CH₃)₃.

The term “C₁-C₄-haloalkoxy”, as used herein, describes C₁-C₄-alkoxy groups as described above where some or all of the hydrogen atoms of these groups are replaced by halogen atoms, i.e., for example, chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy or nonafluorobutoxy.

In the context of the present invention, the term “salts”, of compounds of the general formula (I), is used for the aggregation products of the compounds of the formula (I) with the bases used in step (i) and also, if appropriate, for the aggregation products of mono- or polydeprotonated compounds of the formula (I) with the respective cationic moiety of the bases used in step (i).

Preferably, 1, 2 or 3 of the substituents L¹, L², L³, L⁴ and L⁵ in the compounds of the general formulae (I) and (II) are different from hydrogen. In particular, at least the substituent L¹ is different from hydrogen.

Preferably, the substituents L¹, L², L³, L⁴ and L⁵ in the compounds of the general formulae (I) and (II) are independently of one another selected from the group consisting of hydrogen, halogen, cyano, C₁-C₄-alkyl, C₁-C₄-haloalkyl, C₁-C₄-alkoxy and C₁-C₂-haloalkoxy. In particular, the substituents L¹, L², L³, L⁴ and L⁵ in the compounds of the general formulae (I) and (II) are independently of one another selected from the group consisting of hydrogen, halogen, C₁-C₄-alkyl and C₁-C₄-alkoxy. Particularly preferably, the substituents L¹, L², L³, L⁴ and L⁵ are selected from the group consisting of hydrogen, fluorine, chlorine, bromine, methyl and methoxy. Very particularly preferably, the substituents L¹, L², L³, L⁴ and L⁵ are selected from the group consisting of hydrogen, fluorine, chlorine and bromine.

In a special embodiment of the process according to the invention, L¹, L³ and L⁵ are fluorine and L² and L⁴ are hydrogen.

In the process according to the invention, the heterocyclic compounds of the formula (III) can be employed in the form represented by the formula, in the form of tautomers of these compounds or in the form of tautomer mixtures. If one or more of the groups Y¹, Y² and Y³ are N, the compounds of the formula (III) are frequently also present in the form of the tautomers, as shown in the scheme below using compounds in which Y¹ is N as an example.

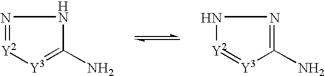

A preferred embodiment relates to a process for preparing a compound of the formula (I) in which Y¹ in the compounds of the general formula (III) and in the tautomers thereof is N and Y² and Y³ are selected from the group consisting of N and CH. Accordingly, a special embodiment relates to a process where in the compounds of the general formula (III) Y¹ and Y³ are N and r is CH. A further special embodiment relates to a process where in the compounds of the general formula (III) Y¹ is N and Y² and Y³ are CH.

Accordingly, suitable compounds of the formula (III) are 1H-pyrrole-2-amine, 1H-imidazole-2-amine, 1H-imidazole-5-amine, 1H-pyrazole-5-amine, 1H-1,2,3-triazole-5-amine, 4H-1,2,4-triazole-3-amine, 1H-1,2,4-triazole-5-amine and also the tautomers of these compounds, such as 1H-imidazole-4-amine, 1H-pyrazole-3-amine, 1H-1,2,3-triazole-4-amine, 2H-1,2,3-triazole-4-amine and 1H-1,2,4-triazole-3-amine, where the compounds mentioned above are unsubstituted or may have a substituent R^{Y} different from hydrogen which is attached to a carbon atom. In particular, the compounds are unsubstituted.

In a particularly preferably embodiment of the process according to the invention, in step (i), the heterocyclic compound of the general formula (III) used is 1,2,4-triazole-5-amine or its tautomer, 1H-1,2,4-triazole-3-amine (amitrole). In this embodiment, very particular preference is given to using 1H-1,2,4-triazole-3-amine.

In a further embodiment of the process according to the invention, two adjacent substituents R^{Y} in the compounds of the general formulae (I) and (III) together are a group —CR^{Y1}═CR^{Y2}—CR^{Y3}═CR^{Y4}— in which the substituents R^{Y1}, R^{Y2}, R^{Y3} and R^{Y4} are each independently of one another selected from the group consisting of hydrogen, CN, NO₂, C₁-C₄-alkyl, C₁-C₄-haloalkyl, C₁-C₄-alkoxy and C₁-C₄-haloalkoxy. In this special embodiment, the substituents R^{Y1}, R^{Y2}, R^{Y3} and R^{Y4} are preferably hydrogen. Examples of such compounds of the formula (III) are optionally substituted 2-amino-indoles or 2-aminobenzimidazoles.

In the process according to the invention, preference is given to using malonic esters of the general formula (II) in which R is C₁-C₄-alkyl, in particular methyl or ethyl. Accordingly, the alcohol of the formula R—OH released during the reaction is a C₁-C₄-alcohol, such as methanol, ethanol, n-propanol, isopropanol, n-butanol or tert-butanol, in particular methanol or ethanol and especially ethanol.

In general, the malonic esters of the formula (II) are employed in an amount of from 0.5 to 2 molar equivalents, preferably from 0.75 to 1.5 molar equivalents, per molar equivalent of the compounds of the formula (III). Especially, the malonic esters of the formula (II) are, based on the compounds of the formula (III), employed in an approximately equimolar amount or in a slightly substoichiometric amount, i.e. from 0.9 to 1.05 molar equivalents of the malonic ester, based on the compound (III).

In the process according to the invention, the alcohol R—OH released by the reaction in step (i) is removed from the reaction solution under reduced pressure. Preferably, the alcohol R—OH is essentially removed completely, i.e. up to a residual content of at most 2% by weight, preferably at most 0.5% by weight and particularly preferably at most 0.1% by weight.

In general, step (i) of the process according to the invention is carried out with the input of thermal energy. The upper limit of the preferred reaction temperature for this step is defined by the boiling points of the compounds of the formulae (II) and (III) employed and by the boiling point of the base used at the respective reaction pressure.

Preferably, the reaction temperature is in a range of from 40 to 250° C., particularly preferably in a range of from 80 to 200° C.

The pressure in step (i) is preferably reduced by distillatively removing, over the entire duration of the reaction, at most 5%, particularly preferably at most 2% and especially at most 1% of the amount of the 2-phenylmalonate of the general formula (II) employed, together with the alcohol R—OH. This is preferably achieved by reducing the pressure during the course of the reaction in a step-wise manner or in particular continually over a relatively long period of time, for example from 2 to 20 h, in particular from 4 to 10 h. Over the entire duration of the reaction, the pressure difference is from 10 to 1000 mbar, in particular from 100 to 990 mbar.

Depending on the temperature, the pressure at the beginning of the process according to the invention is in the range of from 700 to 1100 mbar, in particular 800 mbar to atmospheric pressure, and is reduced during the course of the reaction to a pressure in the range from 5 to 300 mbar, in particular in a range of from 10 to 250 mbar.

The duration of the reaction and the pressure at which the components of a reaction mixture are subject to a change of the state of aggregation depend on the reaction temperature, i.e. if the reaction temperature is increased, firstly the reaction rate is increased and secondly the pressure at which the components of a reaction mixture are subject to a change of the state of aggregation is reduced; if the reaction temperature is reduced, the reaction rate is reduced and the pressure at which the components of a reaction mixture are subject to a change of the state of aggregation is increased. Accordingly, a suitable pressure range for step (i) of the process according to the invention is shown below at a given reaction temperature. Using these data, it will be easy for the person skilled in the art to determine the suitable pressure range at other reaction temperatures using calculations with the aid of generally known physical laws.

At a given reaction temperature of from 120 to 160° C., for example, it is preferred to reduce the reaction pressure continuously or step-wise over a period of from 5 to 10 h from a value of from 1050 to 700 mbar at the beginning of the reaction to a value of from 250 to 50 mbar. Specifically, at a reaction temperature of about 150° C., for example, the reaction pressure is reduced step-wise every hour from a value of about 800 mbar at the beginning of the reaction to a value of about 150 mbar over a period of from 6 to 8 hours. This ensures virtually complete conversion.

In a special embodiment of the process according to the invention, in step (i) the pressure is reduced continuously at a constant temperature.

In the process according to the invention, preference is given to using a base whose boiling point at atmospheric pressure is at least 30° C., preferably at least 50° C. and in particular at least 100° C. above the boiling point of the alcohol R—OH released in the reaction in step (i).

Suitable bases used in step (i) are, for example, tertiary amines. In the context of the present invention, the term “tertiary amine” includes both tertiary amines having at least one tertiary nitrogen atom having three aliphatic or cycloaliphatic substituents which, if appropriate, form a mono- or bicyclic ring skeleton with the nitrogen atom and nitrogen compounds in which the tertiary nitrogen atom is incorporated in an aromatic ring skeleton. The tertiary amines A usually have 1 or 2 tertiary nitrogen atoms, in particular 1 nitrogen atom.

Particularly preferred tertiary amines include at least 6, in particular at least 8 and especially at least 10 carbon atoms, for example 6 to 20, in particular 8 to 18 and especially 10 to 16 carbon atoms. These tertiary amines are characterized in particular by a boiling point which, at the reaction pressure, is at least 5° C., particularly preferably at least 10° C. and very particularly preferably at least 20° C. above the reaction temperature.

Suitable bases for step (i) are, in principle, tertiary amines of the following general formula NR¹R²R³ in which R¹, R² and R³ independently of one another are C₁-C₆-alkyl, C₅-C₈-cycloalkyl, aryl which optionally carries one or two C₁-C₄-alkyl groups as substituents, or phenyl-C₁-C₄-alkyl. The total number of carbon atoms is generally from 6 to 20, in particular from 8 to 18 and especially from 10 to 16. Examples of suitable tertiary amines are N,N-dimethylcyclohexylamine, tripropylamine, tributylamine, N-ethyl-N-propylaminepropane, N,N-dimethylaniline and N,N-diethylaniline, especially tributylamine.

Furthermore suitable for use as base in step (i) are, from among the tertiary amines, pyridine compounds, in particular mono-, di- and tri-C₁-C₄-alkylpyridines having preferably a total of 6 to 18 carbon atoms, such as picolines, mono-, di-, tri(methyl)-pyridines, furthermore phenyl-, pyridyl-, benzyl-, pyridylmethyl- or pyridylethyl-substituted pyridines, furthermore 4-dialkylaminopyridines, and also mono- and di-C₁-C₄-alkoxypyridines.

Furthermore suitable for use as base in step (i) are bridged amines, i.e. tertiary amines in which the amine nitrogen atom is a ring member of a saturated 5- to 8-membered cycle. Examples of these are azabicyclo compounds. Also suitable are saturated 5-, 6-, 7- or 8-membered nitrogen heterocycles which carry a C₁-C₄-alkyl group at the at least one nitrogen atom, such as, for example, N-alkylpyrrolidines, N-alkylpiperidines or N-alkylmorpholines and the like.

Based on one molar equivalent of the malonic ester of the formula (II), the base in step (i) of the process according to the invention is preferably employed in an amount of from 0.1 to 20 molar equivalents. Preference is given to using from 0.75 to 1.5 molar equivalents of the base, based on one molar equivalent of the malonic ester of the formula (II). Based on the malonic ester of the formula (II), the base is employed especially in approximately equimolar amounts or in slightly substoichiometric amounts, i.e. from 0.9 to 1.05 molar equivalents of the base, based on the malonic ester (II).

In a special embodiment of the process according to the invention, the base used in step (i) is simultaneously employed as solvent.

In step (i) of the process according to the invention, a fused dihydroxypyrimidine of the formula (I) or a salt thereof is obtained (X═OH) which may then be converted into the corresponding dichloro or dibromo compound (X═Cl or Br). The conversion may be carried out after work-up of the fused dihydroxypyrimidine of the formula (I) or directly after the reaction in step (i).

The process according to the invention is particularly suitable for being carried out in the form of a “one-pot process”, i.e. the compound obtained in step (i) of the formula (I), in which X is OH, is, without further work-up, employed directly for step (ii). In other words, the reaction mixture from step (i) is used for step (ii) of the process. The reaction mixture from step (i) contains the dihydroxy-substituted compound of the formula (I) in the form of the free compounds and/or the corresponding salts.

In the context of the present invention, halogenating agents are compounds which, under the given reaction conditions, provide a halogen atom, especially chlorine or bromine. Suitable halogenating agents are, for example, POCl₃, PCl₅, POBr₃ or PBr₅, where the reaction with POCl₃ or PCl₅ gives fused pyrimidines of the formula (I), in which X is chlorine, and the reaction with POBr₃ or PBr₅ gives fused pyrimidines of the formula (I), in which X is bromine. Particularly preferably, the halogenating agent used in the process according to the invention is POCl₃.

Step (ii) of the process according to the invention is preferably carried out under superatmospheric pressure. Here, the pressure is preferably in the range between 1 and 15 bar and particularly preferably in the range from 2 to 6 bar.

In a preferred embodiment of the process according to the invention, the halogenation in step (ii) is carried out in the presence of an excess of halogenating agent, especially POCl₃, based on the compound of the formula (I) obtained in step (i) and/or the corresponding salt. Particularly preferably, the halogenating agent, especially POCl₃, is employed in a molar ratio in the range of from 10:1 to 20:1 and very particularly preferably from 13:1 to 17:1, based on the compound of the formula (I) obtained in step (i) and/or the corresponding salt.

In a further preferred embodiment of the process according to the invention, the halogenating agent, especially POCl₃, is initially charged in step (ii) and the compound of the general formula (I) obtained in step (i) and/or the corresponding salt are/is added under the reaction conditions.

After the reaction has ended, the unreacted halogenating agent from step (ii), especially POCl₃, is advantageously removed by distillation. The distillative removal of the halogenating agent, especially POCl₃, is preferably carried out at a temperature of at most 60° C. To ensure an essentially complete distillative removal of the halogenating agent, especially POCl₃, at this temperature, this operation is preferably carried out at a pressure in the range of from 10 to 400 mbar and particularly preferably at a pressure of from 40 to 100 mbar.

The process according to the invention is advantageously suitable for being carried out as a continuous process. Accordingly, the present invention furthermore provides a process according to the invention where at least one of steps (i) or (ii) is carried out continuously. Particularly preferably, at least step (ii) of the process according to the invention is carried out continuously.

In the context of the present invention, the term “continuous process” refers to a process where at least one of the compounds involved in the reaction is continuously fed into the reaction and at least one of the intermediates or products of the reaction is removed continuously in the form of a discharge from a reaction mixture. Specifically, in step (i) the malonic ester of the formula (II) and/or the compound of the formula (III) may be fed continuously to the reaction, and the compound of the formula (I), in which X is OH, may be removed from the reaction mixture. Here, the reaction pressure may be reduced continuously or may be constantly reduced in the continuous process step. In step (ii) of the process according to the invention, the compound of the formula (I), in which X is OH, especially as a discharge from a continuously executed process step (i), and/or the halogenating agent may be added continuously. Preferably both are added such that there is a significant excess of halogenating agent in the reaction mixture at any point in time of the continuous process. The compound of the formula (I), in which X is Cl or Br, is removed in the form of a reaction mixture and subjected to a separation. The starting materials and intermediates obtained during the separation of reaction mixtures may advantageously be recycled into the process steps in question. Suitable reactors for continuous reaction are known to the person skilled in the art and described, for example, in Ullmanns Enzyklopädie der technischen Chemie [Ullmann's Encyclopedia of Industrial Chemistry], Vol. 1, 3. ed., 1951, p. 743 ff.

For the continuous production of compounds of the general formula (I), in which X is halogen, the halogenating agent and the compound of the general formula (I), in which X is OH, are preferably mixed cold, i.e. below the reaction temperature, and this mixture is fed into step (ii) of the process with simultaneous discharge of a reaction mixture comprising the compound of the formula (I), in which X is halogen.

Hereinbelow, the process according to the invention is illustrated by non-limiting examples.

### EXAMPLES

### Example 1

### Preparation of 6-(2,4,6-trifluorophenyl)[1,2,4]triazolo[1,5-a]pyrimidine-5,7-diol or the corresponding salt (at reduced pressure)

At 25° C., diethyl 2-(2,4,6-trifluorophenyl)malonate (290.2 g, 1.0 mol), tributylamine (185.4 g, 1.0 mol) and 3-amino-1,2,4-triazole (85.8 g, 1.02 mol) are combined. The pressure in the stirring vessel is reduced to 800 mbar and the reactor content is then heated to 150° C. During heating to reach this temperature, some of the ethanol released in the reaction is already distilled off. After an internal temperature of 150° C. is reached, the reaction mixture is stirred at 150° C. for 7 h. During this time, the pressure in the reaction vessel is set as follows: initially 1 h at 800 mbar, then 1 h at 650 mbar, 1 h at 500 mbar, 1 h at 400 mbar, 1 h at 300 mbar and finally 2 h at 150 mbar. In this manner, the ethanol released during the reaction is distilled off almost completely. This gave a viscous solution (463.8 g) which, according to quantitative HPLC analysis, had a product content of 59%. This corresponds to a yield of 97%.

### Comparative Example 1

### Preparation of 6-(2,4,6-trifluorophenyl)[1,2,4]triazolo[1,5-a]-pyrimidine-5,7-diol or the corresponding salt (at atmospheric pressure)

At 25° C., diethyl 2-(2,4,6-trifluorophenyl)malonate (290.2 g, 1.0 mol), tributylamine (185.4 g, 1.0 mol) and 3-amino-1,2,4-triazole (85.8 g, 1.02 mol) are combined. At atmospheric pressure, the reactor content is heated to 150°. During heating to reach this temperature, some of the ethanol is already distilled off. After an internal temperature of 150° C. is reached, the reaction mixture is stirred at 150° C. for 7 h. During this time, further ethanol is distilled off. This gave a viscous solution (451.6 g) which, according to quantitative HPLC analysis, had a product content of 56%. This corresponds to a yield of 89.8%.

The reaction discharge comprised 2.2% of unreacted diethyl 2-(2,4,6-trifluorophenyl)-malonate and about 2% of ethanol.

### Example 2

### Preparation of 5,7-dichloro-6-(2,4,6-trifluorophenyl)[1,2,4]triazolo[1,5-a]-pyrimidine

Step (i): at room temperature, diethyl 2-(2,4,6-trifluorophenyl)malonate (99.5% pure, 240.6 g, 0.825 mol), 3-amino-1H-1,2,4-triazole (amitrole, 98.3% pure, 70.6 g, 0.825 mol) and tributylamine (153.5 g, 0.825 mol) are initially charged in a stirring vessel with distillation bridge. The pressure in the stirring vessel is reduced to 800 mbar, and the reactor content is then heated to 150° C. During heating to reach this temperature, some of the ethanol released in the reaction is already distilled off. After an internal temperature of 150° C. is reached, the reaction mixture is stirred for 7 h at an internal temperature of 150° C. and the following pressure: initially 1 h at 800 mbar, then 1 h at 650 mbar, 1 h at 500 mbar, 1 h at 400 mbar, 1 h at 300 mbar and finally 2 h at 150 mbar. The ethanol released during the reaction is distilled off almost completely.

Step (ii) (at atmospheric pressure): at 150° C. the reaction mixture, obtained in the form of a viscous oil, is transferred into a heatable dropping funnel and, over a period of 17 minutes, added dropwise with stirring to POCl₃ (1897 g, 12.38 mol) at a temperature of from 100° C. to 107° C. After the addition has ended the reaction mixture is stirred under reflux at a temperature of from 107 to 115° C. for a further 10 hours. Excess POCl₃ (1575.3 g) is removed by distillation under reduced pressure (200 mbar) at a temperature of from 60 to 105° C. After the distillation has ended, toluene (420 ml) is added to the distillation residue, which is then cooled to 40° C. The resulting solution is then, at 20 to 26° C., added with stirring to a mixture of toluene (420 ml) and water (1048 ml). After heating to 50° C. and accomplished phase separation, part of the organic phase (5-15%) is distilled off at a pressure of 200 mbar. This gave 1006 g of a toluene solution which, according to quantitative HPLC analysis, contained 20.9% of the desired compound. The yield for the chlorination step was 85.2%. This corresponds to a yield of 82.4% over both synthesis steps.

### Example 3

### Preparation of 5,7-dichloro-6-(2,4,6-trifluorophenyl)-[1,2,4]triazolo[1,5-a]-pyrimidine

Step (i): at room temperature, diethyl 2-(2,4,6-trifluorophenyl)malonate (99.8% pure, 572.9 g, 1.98 mol), 3-amino-1H-1,2,4-triazole (amitrole, 98.3% pure, 170.7 g, 2.0 mol) and tributylamine (366.3 g, 1.96 mol) are initially charged in a stir vessel with distillation bridge. The pressure in the stirring vessel is reduced to 800 mbar, and the reaction mixture is then heated to 150°. During heating to reach this temperature, some of the ethanol released in the reaction is already distilled off. After an internal temperature of 150° C. is reached, the reaction mixture is stirred for 7 h at an internal temperature of 150° C. and the following pressure: initially 1 h at 800 mbar, then 1 h at 650 mbar, 1 h at 500 mbar, 1 h at 400 mbar, 1 h at 300 mbar and finally 2 h at 150 mbar. The remaining ethanol released during the reaction is distilled off almost completely.

Step (ii) (under elevated pressure): as a melt, the reaction mixture obtained in this manner is, at a temperature of 150° C., added via a heat-traced pipe from the first stirred vessel to POCl₃ (4875.9 g, 31.49 mol) initially charged at 25° C. in a further vessel (HC pressure stirring vessel). The pressure stirring vessel is then closed and heated to 140° C. This generates a pressure of about 2.4 bar. After a further 1.5 h, the reaction has ended. During this period of time, the pressure increases to about 2.6 bar. The content of the pressure vessel is cooled to 25° C., the pressure falling to about 0.15 bar. The pressure vessel is then vented slowly. Excess POCl₃ is distilled off under reduced pressure (100 mbar) and a temperature of up to 60° C. At 60° C., the pressure is then reduced step-wise to 40 mbar. A total of 4005 g of POCl₃ distillate are obtained which can be used in the next experiment instead of fresh POCl₃. The distillation residue is dissolved by addition of toluene (955.7 g). This gives about 2620 g of solution. In a further stirring vessel, water (2747.8 g) and toluene (960.5 g) are initially charged. The toluene solution of the distillation residue (2620 g) is then added at a temperature of 50° C. over a period of 2 to 3 h. Separation of the phases at a temperature of 50° C. gave 2527.8 g of the toluene phase. According to quantitative HPLC analysis, this phase contained 22.7% of 5,7-dichloro-6-(2,4,6-trifluorophenyl)-[1,2,4]-triazolo[1,5-a]pyrimidine. This corresponds to a yield of 93.6% for step (ii) and 90.8% over steps (i) and (ii) of the process according to the invention.

### Comparative Example 2

### Preparation of 5,7-dichloro-6-(2,4,6-trifluorophenyl)[1,2,4]triazolo[1,5-a]pyrimidine

Step (i) (not according to the invention): at room temperature, diethyl 2-(2,4,6-trifluorophenyl)malonate (96% pure, 226.7 g, 0.75 mol), 3-amino-1H-1,2,4-triazole (amitrole, 96% pure, 65.7 g, 0.75 mol) and tributylamine (139.5 g, 0.75 mol) are initially charged in an apparatus with distillation bridge, with stirring, the mixture is heated to 150° C. (with nitrogen being bubbled through the mixture) and heated at 150° C. for 6 hours. This gives about 61 g of distillate.

Step (ii): the reaction mixture obtained (viscous oil) is cooled to 120° C., and at a temperature of from 120 to 130° C. phosphoryl chloride (689.9 g, 4.5 mol) is then added over a period of about 1 h. After the addition has ended, the mixture is stirred at 125° C. (reflux) for 7 hours. Phosphoryl chloride is distilled off at 130 to 135° C. After the distillation has ended, the distillation bottom (crude dichlorotriazolopyrimidine) is cooled to 100° C. and, at 40 to 50° C., added dropwise to a mixture of 838 ml of toluene and 1048 ml of water. After 30 minutes of stirring at 50° C., the phases are separated. The organic phase contains 19.4% of the desired dichlorotriazolopyrimidine (=179.6 g). Yield over both synthesis steps: 74.9%. Yield for the chlorination step: 78.9%.

1-15. (canceled)

16. A process for preparing aryl-substituted fused pyrimidines of the general formula (I), 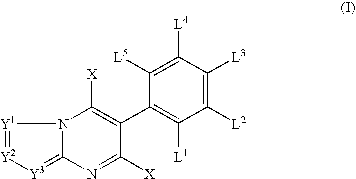
in which
L¹, L², L³, L⁴ and a L⁵ independently of one another are hydrogen, halogen, cyano, nitro, C₁-C₄-alkyl, C₁-C₄-haloalkyl, C₁-C₄-alkoxy, C₁-C₄-haloalkoxy, C₁-C₄-alkylcarbonyl, C₁-C₄-haloalkylcarbonyl, C₁-C₄-alkoxycarbonyl, C₁-C₄-alkylaminocarbonyl or di-(C₁-C₄-alkyl)aminocarbonyl and
Y¹, Y², Y³ independently of one another are C—R^{Y} or N,
where the substituents R^{Y} independently of one another are selected from the group consisting of hydrogen and C₁-C₄-alkyl which is optionally mono- or polysubstituted by halogen, cyano, nitro, C₁-C₄-alkoxy, C₁-C₂-haloalkoxy, C₁-C₄-alkylaminocarbonyl or di-(C₁-C₄-alkyl)aminocarbonyl; or
where two adjacent substituents R^{Y} together with the atoms to which they are attached form an aromatic or partially saturated, optionally substituted 5- to 7-membered ring; and
X is hydroxyl, chlorine or bromine;
which comprises
(i) reacting a 2-phenylmalonate of the general formula (II), 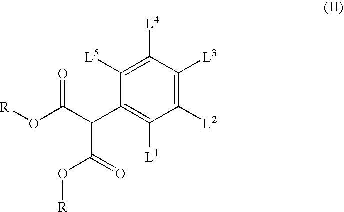
in which R is C₁-C₈-alkyl and the substituents L¹, L², L³, L⁴ and L⁵ have one of the meanings given above
with a heterocyclic compound of the general formula (III) or a tautomer thereof, 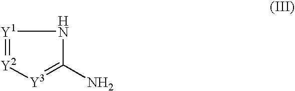
in the presence of a suitable base,
where an alcohol, released during the reaction, of the formula R—OH is continuously removed from the reaction mixture under reduced pressure;
giving a compound of the formula (I) or a salt thereof in which X is OH,
and, if X in the compound of the formula (I) is chlorine or bromine,
(ii) reacting the compound of the formula (I) or the salt obtained in step (i) with a halogenating agent.

17. The process of claim 16 where in step (i) 3-amino-1H-1,2,4-triazole is used as tautomer of the heterocyclic compound of the general formula (III).

18. The process of claim 16 where R in formula (II) is methyl or ethyl.

19. The process of claim 16 where in step (i) the alcohol R—OH is removed to a residual concentration of at most 1% by weight.

20. The process of claim 19 where in step (i), at constant temperature, the pressure is reduced continuously.

21. The process of claim 16 in which the base is selected from tertiary amines comprising at least 6 carbon atoms.

22. The process of claim 21 in which the base is tributylamine.

23. The process of claim 16 where the substituents L¹, L², L³, L⁴ and L⁵ in the compounds of the general formulae (I) and (II) independently of one another are hydrogen, fluorine, chlorine or bromine.

24. The process of claim 16 where 1, 2 or 3 of the substituents L¹, L², L³, L⁴ and L⁵ in the compounds of the general formulae (I) and (II) are different from hydrogen.

25. The process of claim 16 where the compound of the formula (I) in which X is OH and/or the corresponding salt are/is used in the form of the reaction mixture obtained in step (i) for step (ii) of the process.

26. The process of claim 16 where the reaction in step (ii) is carried out at a pressure in the range of from 2 to 6 bar.

27. The process of claim 16 where the halogenating agent, based on the compound, obtained in step (i), of the formula (I) and/or the corresponding salt is employed in a molar ratio of from 13:1 to 17:1.

28. The process of claim 16 where in step (ii) the halogenating agent is initially charged and the compound, obtained in step (i), of formula (I) and/or the corresponding salt are/is added under reaction conditions.

29. The process of claim 16 where unreacted halogenating agent is removed by distillation after the reaction has ended.

30. The process of claim 16 where the halogenating agent is POCl₃.
